(19) United States
(12) Patent Application Publication
(10) Pub. No.: US 2026/0285679 A1
(43) Pub. Date: Sep. 24, 2026

(54) PROTON RICH IONIC FLUID AND METHODS OF TEST-AFFIRMATION

(71) Applicant: TegIpco, LLC, Dover, DE (US)

(72) Inventors: Nicholas Joseph Wilson Arvanitakis, Cochise, AZ (US); Chrisanthos Arvanitakis, Cochise, AZ (US)

(73) Assignee: TegIpco, LLC, Dover, DE (US)

(21) Appl. No.: 19/059,552

(22) Filed: Feb. 21, 2025

(51) Int. Cl. C01B 3/08; B65B 5/04; G01N 30/86; G01N 33/00

(52) U.S. Cl. CPC C01B 3/08; B65B 5/04; G01N 30/8675; G01N 33/004; G01N 33/005

(57) ABSTRACT

A testing-affirmation for specific properties of a proton-rich ionic fluid is disclosed. This testing requires reforming the fluid into H2 gas, using a test-reformer. Specific embodiments of reformers suitable for use in actual production of energy are also disclosed. These will be referred to as production-reformers to distinguish them from test-reformers. Numerous practical usages including combining a proton-rich ionic fluid (PRIF) with gas with liquid fuels e.g. jet fuel or diesel fuel. Another usage combines a proton-rich ionic fluid (PRIF) with gas with liquid fuels e.g. jet fuel or diesel fuel.

### BACKGROUND OF THE INVENTION

There is a significant need for production of hydrogen that can be achieved with less consumption of energy to isolate the hydrogen. However, a long-time problem is that many entities claiming to achieve this are later found to be not credible. Consequently, a trustworthy and reliable way of accurately testing production of hydrogen is desired.

### SUMMARY OF THE INVENTION

This disclosure will discuss testing-affirmation specific properties of a proton-rich ionic fluid. This testing requires reforming the fluid into H2 gas, using a test-reformer. However, this disclosure will also discuss specific embodiments of reformer suitable for use in actual production of energy, which will be referred to as production-reformer to distinguish it from a test-reformer. For example, one practical usage of the embodiments herein is the known practice of combining H2 gas with liquid fuels e.g. jet fuel or diesel fuel. While these practices are known, they have disadvantages, and people claiming to enhance/augment the production of hydrogen are often found to be not credible. The embodiments herein overcome some of these disadvantages.

This disclosure will be divided into two main parts: a) various proprietary ways of accurately attesting and affirming valid hydrogen production, and b) various proprietary reformers to assist in putting that produced hydrogen to good use.

### BRIEF DESCRIPTION OF THE DRAWINGS

The various embodiments of the inventive subject matter of the present disclosure will be described in more detail in conjunction with the following figures. The structures in the figures are illustrated schematically, and they are not necessarily drawn to scale. The figures are not intended to show actual dimensions.

FIGS. 1A and 1B show non-limiting arrangements of reactor systems for producing a Proton-Rich Ionic Fluid (PRIF) according to the embodiments herein;

FIGS. 2, 3A, and 3B show example methods of operation of the reactor systems of FIGS. 1A and 1B;

FIGS. 4A, 4B, 4C, 5A, 5B, 5C, 5D, 6A, 6B, and 6C show detail of one or more recirculators;

FIGS. 7A-7B-7C show contrasting arrangements of alternate embodiments of reactor systems;

FIG. 7D shows a recirculator using information from a testing module;

FIGS. 8A-8B are graphs comparing fuel potential inside a wing tank, while factoring inefficiencies and losses;

FIG. 9 shows the same energy density comparisons as FIGS. 8A-8B, asserting all the same data but using separate measurement units along the axes;

FIG. 10 shows a portable manufacturing system (PMS) 1004 which converts water into PRIF using some form of the proprietary electromagnetic processes shown in FIGS. 1-7;

FIG. 11 shows a summary of properties of the PRIF;

FIG. 12 is a chart regarding fuel-safety at airports;

FIG. 13A shows a simplified reformer;

FIG. 13B conveys that the embodiments herein can affirm and verify that hydrogen gas is captured at the both anodes and cathodes of the reformer;

FIG. 13C is an analysis of all gases produced by a reformer;

FIG. 14 shows an experiment in which PRIF as added to a sample crude oil, and then performing SARA (Saturated, Aromatics, Resins, Asphaltenes) testing on the resulting combination;

FIG. 15 shows a variety of portable reformer-environments, and includes an actual reformer;

FIG. 16A show an example reformer;

FIG. 16B shows that the reforming is not linear and instead exponential;

FIG. 17 conveys that the PRIF also has its own quasi-Grothuss type of behavior; and

FIG. 18 shows the steps in computing amount of energy needed for creation of 1 kg of H2 gas from the PRIF.

### DETAILED DESCRIPTION OF THE PREFERRED EMBODIMENTS

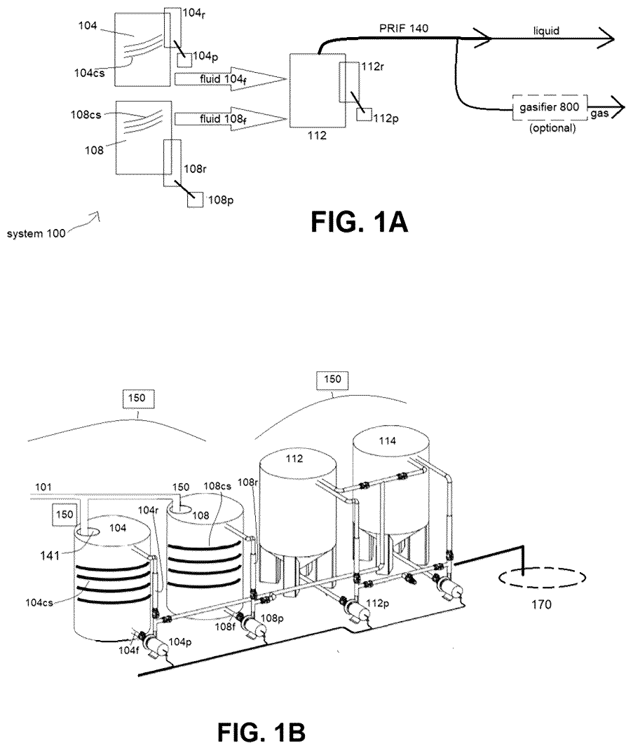

FIG. 1A shows an example system 100 for producing a proton-rich PRIF (PRIF) 140. The system 100 converts a common hydrogen-based input fluid 101 to the PRIF 140 comprising an overabundance of hydrogen H₁+ atoms, mainly just protons since atomic hydrogen does not have a neutron and the electron has been peeled off. This conversion occurs in the absence of elevated temperatures or pressures, so that the resulting PRIF 140 is suitable for shipping or storage at Standard Temperature and Pressure (STP, AKA Normal Temperature and Pressure NTP). One example period of reliable shelf-life of the PRIF 140 might be 36 months, although there could be examples of even longer shelf-life, depending on the specific formulation.

The input fluid 101 may be one of various commonly-found hydrogen-donating fluids or mixes of multiple hydrogen-donating fluids, and can also be dirty water, fracked water, and/or processed water. A non-limiting list of potential types of hydrogen-donating fluids can be found in an Appendix A to this disclosure, titled “EXAMPLE hydrogen-donating input fluids 101”.

Referring to FIGS. 1A and 1B, an example system 100 and flowchart includes a first tank 104, a second tank 108, a third tank 112, and corresponding recirculators 104r, 108r, 112r. Both first and second tanks 104\108 comprise recirculator 104r\108r, pump 104p\108p, and windings or inductor coils 104cs\108cs. Both first and second tanks also pump out intermediate fluids 104f\108f that has been partially-processed and is on its way to becoming the proton rich ionic fluid (PRIF) 140. FIG. 1B shows a fourth tank 114 which acts as a potential overflow tank, or storage tank, or other way of assisting in management of PRIF 140 during or after a production run thereof. In the flowcharts of FIGS. 1A-1B, all activity flows from left to right.

The tanks 104\108 have the circumferential windings 104cs\108csapplied to their outer surface thereby forming a reaction zone. The windings 104cs\108cs can be formed with stranded wire or other types of windings to act as a large-scale inductor coil. FIG. 1B also shows a seal 141 on the tank, and a detector 150. The tanks 104/108/112 can be operated at NTP/STP, but for detecting various gaseous components, the seal 141 could be helpful in trapping and capturing. The detector 150 can capture a lot of different components, as will be discussed in more detail herein.

The circumferential windings or inductor coils 104cs\108cs may be electrically coupled to a power supply so as to be electrically coupled to either alternating or direct current at a variety of frequencies. An amount of insulation on the wires and tanks, spacing between specific windings, and wire gauge all may vary according to a desired outcome.

The pumps 104p\108p are coupled to the recirculators 104r\108r which have magnetic modules 508 in various orientations attached thereto. However, the magnetic modules 508 can come in a lot of widely differing formats, of which the embodiments shown in the various FIGS herein are but non-limiting examples.

The activity within the reactor system(s) 100 result in removing electrons from the input fluid in such a way that the resulting PRIF becomes electron-deficient. This PRIF 140 can remain electron deficient at STP for varying periods, e.g. having a shelf-life of 36 months.

The circumferential windings 104cs\108cs can have a variety of voltages and currents applied thereto. The voltage applied to the windings 104cs may be equal to that applied to the windings 108cs, or may not. Further, a voltage may be applied to one set of windings but not the other, and polarity may be altered.

A pre-determined wattage for the circumferential windings 104cs\108cs can be selected based on the chemical constituents of the input fluid 101, a desired configuration of the PRIF 140, ambient temperature, volume of end-product, and other factors. As current moves through windings 104cs\108cs, a corresponding magnetic field directed perpendicularly to windings 104cs\108cs applies a magnetostatic force to liquid 101 while being circulated through the tanks 104\108 for a predetermined period of time until the outlet fluid 104f\108f is transferred via e.g. to the 3^{rd} tank 112.

The magnetostatic forces applied to the windings 104cs\108cs can be adjusted between 2,000-80,000 Gauss, with 20,000-80,000 Gauss being a preferred range. When outlet openings 104f and 108f are opened, the fluids 104f\108f are combined into the third tank 112 which comprises a recirculator 112r and pump 112p. Once the fluid from both first tank 104 and second tank 108 are combined into the third tank 112, the combination is pumped and recirculated within the third tank 112.

Unlike the first tank 104 or second tank 108, third tank 112 does not have a circumferential windings, and therefore experiences no electrostatic effects. Instead, the third tank 112 experiences an oscillating magnetic field through the recirculator 112r due to the magnetic-modules 508 attached thereto.

During operation of the system 100, some oxygen vapes off, and goes away in a variety of forms. This is due to the fact that one purpose of the system 100 is to break the covalent bonds of a water molecule, separate out the oxygen\electrons and drive them off (prevent them from re-combining), and thus isolate protons in the form of H₁+. One reason this can be done at low power is because a typical water molecule is known to be a weak dipole, where some of the H can be separated from the O just by mechanical forces, some of which occur within the recirculators 104r/108r/112r.

The sensors 150 are used to affirm proper performance of the system 100, including temperature. In tank 104 there may be a slight exotherm 20-30 degrees F based on which proton donor was used within the input fluid 101. Content of the specific chosen input fluid 101 can affect this, due to clean water v. dirty water v. produced water or other type of effluent source (see Appendix A).

Oxygen may gas off maybe 2-3% in overall mass difference, perhaps in the format of O2 but also in other formats. Various oxygen radicals are formed during production-use of the system 100, mostly oxygen based salts, which can vary according to a wide variety of conditions including but not limited to the content of the input fluid 101. These salts end up getting excreted through the back-end portion 170 of the system 100.

In a lower-cost embodiment, the detector 150 can be focused mainly on CO2 and O2, which both have special significance in hydrogen generation. However, the detector 150 can have wider scope, depending on manufacturing considerations and end-customer preferences.

If the input fluid 101 contains sulfuric acid, that can lead to sulfate salts, colloidal sulfur, and/or sulfur dioxide. Meanwhile, produced water tends to result in carbonates, oxides, and chloride salts. Acetic acid can lead to acetate salts.

The semicircle 170 represents a combination of filters, precipitate catch mechanisms, and or hydrocyclone, which may catch any of the below. That is, a non-limiting list of specific oxygen radicals and salts (either gas or solid) given off during use of the system 100 can include but are not limited to:
hydroxide salts (_OH); carbonate salts (_CO3); sulfate salts (_SO4); nitric salts (_NO3); dioxides (_O2), the most of important of which is CO2; acetates (_CH3COO—); and Alkoxides (_COH alcohol salts).

The proton-donating input fluid 101 (Appendix A) can comprise many different blends and even different waters and oils thus any of these will have different sludges and precipitates.

FIGS. 2 and 3A-3B show example methods of operation of the reactor systems of FIGS. 1A and 1B. Regarding the flowchart of FIG. 3A, in an embodiment, the second tank 108 might have twice the capacity of the first tank 104. An example operation of the flowchart of FIG. 3A might be where the tanks 104/108 are filled up with the input fluid 101 in equal proportions, and processed separately. The recirculators 104r/108r (not shown in FIG. 3A) could be set to opposite polarities. Then, the contents of tank 104 could be put into second tank 108 for further processing for predetermined time periods.

The second tank 108 might have the following elements added which may not be in the first tank 104: flocculants, polyacrylamides, ferric sulfates, and/or gypsum. An additional variation might be to add alcohol to the input of the first tank 104.

FIGS. 4A, 4B, 4C, 5A, 5B, 5C, 5D, 6A, 6B, and 6C show detail of the recirculators 104r, 108r, and 112r, which are sometimes referred to as static mixers. As shown at least within FIGS. 4A-4C, each recirculator can be formed as an elongated translucent tube that has movable internal fluting 404 (AKA baffle) located therein. The recirculators 104r, 108r, and 112r further comprise a grommet 420 at each end, along with threaded surfaces so that they may be connected in series. The internal fluting 404 aids in restraining fluid flowing through the tubes 416 thereby forming a type of reaction zone in which covalent bonds can be broken, and heterolysis can occur (FIG. 2). Each internal fluting 404 can be formed with a plurality of grommets 420 that can be concatenated to one another so as to form a chain structure if desired. The fluting 404 is important because it can break or at least strain the covalent bonds holding water together. It is an advantage of the embodiments herein to break the covalent bonds of the water with as little energy as possible. The fluting 404 leverages the fact that water molecule has weak dipole, a weak covalent bond.

FIG. 4B shows example windings 424 and inductor coils 428 embedded within the plexiglass body (tube 416) of a recirculator. These coils 428 are configurable at a variety of polarities and electromagnetic capabilities. FIG. 4C shows another example of inductor-patterning, where an inductive mechanism 432 is configured in a “rear window defogger” serpentine configuration.

FIG. 5A shows an example recirculator with magnets 509 taped on. FIG. 5B shows an example of rectangular magnet 509 that is polarized in a way different than a domino magnet. FIG. 5C shows a recirculator with a slidable adjustable mechanical magnet-cuff 460. FIG. 5D shows a recirculator with a slidable adjustable electrical inductor-cuff 460.

The system 100 is designed to work in a variety of locations and climates, and with widely varying quality of water including unknown salinity, unknown metal content, unknown viscosity, and unknown level of pollutants. Accordingly, the magnetic modules 508 would be tunable and subject to continual adjustment. The system 100 may be used in remote areas where spare parts may be inaccessible, and may receive what small amounts of power it needs, from solar devices or off-grid devices that have varying levels of reliability. The magnetic modules 508 will have a lot of flexibility and adjustability, both mechanically and also electronically.

Moving to FIG. 5A, within any particular recirculator, the plurality of magnetic modules 508 are arranged circumferentially about the outer surface of the tube 416 and periodically located its length. In some embodiments, a magnet pack 508 is formed with one or more static bar-magnets 509 that define opposite polarities often denoted as a North and South.

The magnetic modules 508 are arranged on an outer surface of the tube 416 in specific ways. One example arrangement is where each North pole side may be facing e.g. radially inwardly, toward the center of tube 416. In this arrangement, each South pole side of a magnet or magnet group 509 would then face radially outwardly from an outer surface of the tube 416. The specific size, shape, and orientation of the individual magnets 509 can vary. FIG. 5B shows an example magnet 509 having a non-domino shape, but that is for example only.

As shown in FIG. 2, in operation, input fluid 101 is piped into tanks 104\108 until at least partially filled. The tanks 104\108 will have a predetermined wattages applied through their respective windings 104cs\108cs for predetermined time periods, often at least 45 minutes. Often, current applied through the circumferential windings 104cs\108cs may be between 5-100 amps at a wattage between 60-1200 watts, with 100 amps at 1,000 watts being advantageous. FIG. 3B shows another way of interpreting the flow within the system 100

During use, the recirculating pumps 104p\108p move the input fluid 101 through the tanks 104\108 via the recirculators 104r\108r. These in turn apply a uniform static magnetic field to input liquid 101 via the magnets 508.

A polarity applied to the recirculator 104r may be opposite the polarity applied recirculator 108r. In one embodiment, recirculator 104r will be set with North pole sides 193 facing radially inwardly applying a total of 46,000 Gauss to input liquid 101, while the recirculator 108r will be set with South pole sides facing radially inwardly thereby applying a total of 46,000-58,000 gauss to the input liquid 101.

Continuing this example, constant recirculation of the input fluid 101 from the tanks 104\108 through recirculators 104r\108r causes a non-transitory polar imbalance in the input liquid 101 resulting from breaking the weak dipole known to be present in water. The differences in fluid velocities within recirculators 104r\108r thus creates a separation and segregation of atomic hydrogen H₁+ within the input fluid 101.

The reactor system(s) 100 can be operated with a variety of ranges and thus have a lot of configurability and ability to be customized for specific types of production runs of the PRIF 140, and also can be adapted to specific types of input fluid 101. As stated, typically, the input fluid 101 will be a hydrogen-donating fluid such as shown in Appendix A. Further, each of the first, second, and third recirculators 104r\108r\112r can separately apply a pre-configured magnetic field to the fluid circulating therein, therefore creating a separate proton-rich vortex within each of the plurality of tanks 104\108\112. These pre-configured magnetic fields can be adjusted applied by the recirculators can be auto-adjusting. Further, if the right levels of intermediate fluids 104f\108f are occurring, the magnetic fields can be shut off entirely.

The specific magnetic field applied may vary according to characteristics of the input fluid 101. A key factor is that heterolysis (FIG. 2) occurs and breaks the covalent bonds in the water-portions of the input fluid. Subjecting the input fluid 101 to a magnetic field provides a low-cost non-CO₂-creating way of doing this.

FIGS. 6A-6C show example recirculators 104r/108r/112r and FIG. 6C shows a testing module 704 that can affect production of the PRIF 140 in real-time. Under the right circumstances, the inductors of FIG. 6C can be re-oriented in a variety of patterns and polarities, hence the question-marks of FIG. 6C. The recirculator of FIG. 6C is patterned to look similar to FIG. 6B, which shows static magnets with known fixed polarities, but that is for illustration-only and the embodiments herein should not be considered as limited exclusively thereto. Instead, FIG. 6C should be interpreted to borrow from the example of FIG. 6B, but expand it to show a variety of configurations and adjustable features including not being committed to a specific polarity. The embodiment of FIG. 6C shows a test module 704 and columns of magnetic modules 508 that can be changed depending on feedback from the test module.

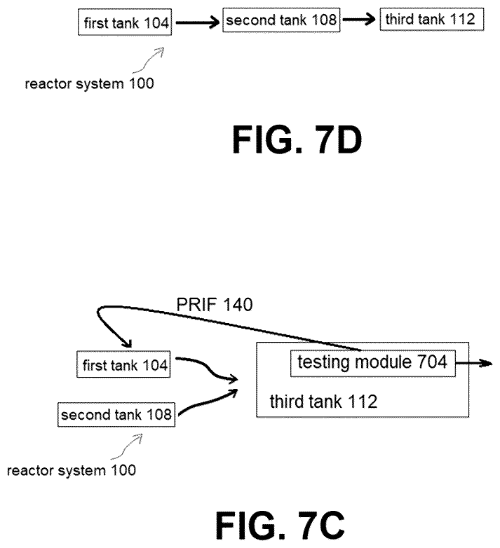

The testing module 704 of FIG. 6C and FIG. 7D can sense breaking of covalent bonds, other factors, and can adjust magnetic or electromagnetic fields and polarities in order to achieve a desired content of PRIF 140.

The testing module 704 can comprise a mass gas analyzer, ammonia or peroxide analyzers, and potentially API testing. API testing can include high-resolution mass spectrometry, liquid chromatography, high-performance thin-layer chromatography (HPTLC), and stability testing.

FIGS. 7A-7B-7C show contrasting arrangements in which potential alternate embodiments of the system 100 can include a 2-tank rather than 3-tank system 100. FIG. 7D shows another alternative routing within the system 100 including the testing module 704 that may optionally make decisions on sending fluid back to earlier tanks for further processing.

### FAKE GREEN HYDROGEN

The expression “Fake green hydrogen” refers to a situation where a company or entity claims to be producing “green hydrogen” meaning where the process requires minimal energy and gives off minimal CO₂ but is actually generating it through e.g. fossil fuels. This is essentially misleading consumers about the true sustainability of their product and also the amount of CO₂ given off. Another expression is “greenwashing” of hydrogen production.

Companies sometimes label hydrogen produced from fossil fuels as “green” to appear more environmentally friendly, to gain tax advantages, and to not reveal the amount of CO2 given off.

It can be difficult to confirm whether a company is truly producing green hydrogen as advertised. Most hydrogen is produced in the form of H2 gas that is produced by electrolysis. Certification entities are sometimes employed to provide verification and assurance that the company is using verifiable renewable energy sources to power their electrolysis process.

In sharp contrast, when using the PRIF 140, there is no electrolysis. Second, there is no CO₂ given off. The various CO₂ monitors 150 shown in e.g. FIG. 1B would affirm that. Further, the PRIF 140 is single H₁+ not H2 gas, thus does not require cracking the H2 gas.

There exists another factor in affirming authentic green Hydrogen, meaning truly green and not “astroturf” or artificially green. This factor involves proving out that the H2 gas was not even partially derived from SMR and Haber Bosch processes, as these both produce huge amounts of CO2. This is sometimes referred to as greenwashing. In order to seem more green, some entities hide their base-origins and hide the amount of coal burned to produce the hydrogen.

### CO2 MEASUREMENTS

To address this, the system 100 features CO2 sensors 150 embedded at numerous locations within the system 100.

It is difficult to accurately measure gas contaminants. However, a single analyzer 150 for multiple natural gas contaminants can achieve accurate and reliable measurement. If necessary, the tanks can use the seal 141 to have an accurate inventory of everything given off within that specific tank. Further, the test data can be transferred in a tamper-proof way that cannot be overwritten, which is helpful for affirming authentic green hydrogen. The gas analyzer 150 is introduced mainly for CO2 detection, but can be used for many other purposes as well.

### SEMANTICS OF JET FUEL

Within this disclosure, the expression “JP-8” will sometimes be used, other times “Jet-A”. While these are not exactly the same, within this disclosure their purpose is the same, such that one will be considered interchangeable with the other. This ends the section on semantics.

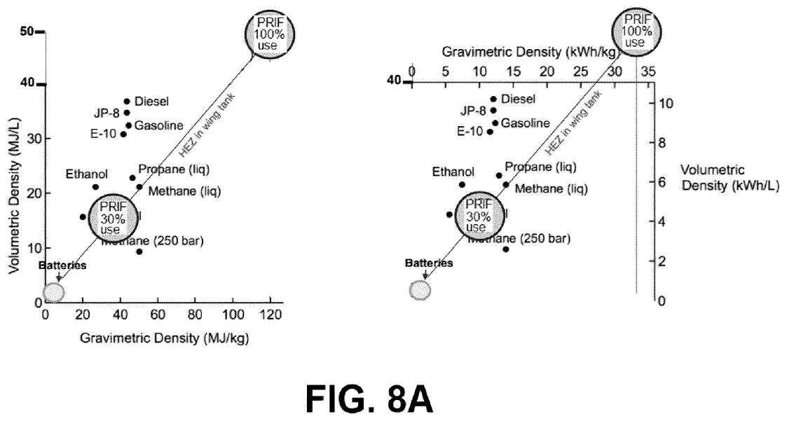

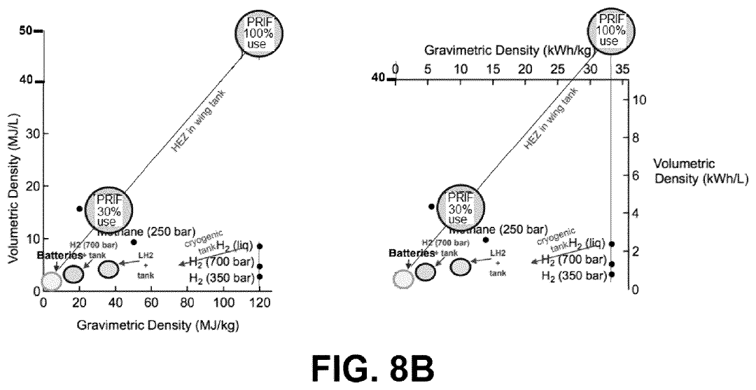

FIGS. 8A-8B (side by side) compare PRIF 140 to a variety of fuels including diesel or gasoline where their horizontal axis is gravimetric and the vertical axis is volumetric. The most optimal fuels would be those that are located as far as possible to the upper right.

The LH side and RH side of FIGS. 8A-8B are exactly the same data, but their horizontal & vertical axes are re-located to make the data easier to read. Further, FIGS. 8A-8B are also split into two Figures because the text-data and positioning is so dense and congested, that it's difficult to digest when all is in the same single chart.

It's very unusual for any fuel to burn at 100% efficiency with absolutely no emissions. While the PRIF 140 is efficient, the differing efficiencies of 30% and 100% are plotted within FIGS. 8A-8B to maintain credibility. It is important to not make absurd or unreasonable assertions about the PRIF 140.

FIGS. 8A-8B convey even if only utilizing 30% of the total amount of PRIF 140, with the rest being lost, the PRIF energy capability is still greater than all current hydrogen offerings, even higher than methane.

The PRIF 140 is 35% denser than Jet A and is non-toxic, yielding 35% more power than Jet A. Diesel and other liquid fuels are often burned at around 30% efficiency, so FIGS. 8A-8B model the PRIF 140 on those criteria as well, to be consistent. However, the PRIF 140 will be shown in this disclosure to have a much higher efficiency than 30%.

FIGS. 8A-8B are is helpful in comparing fuel potential inside a wing tank, while factoring inefficiencies and losses. Looking at PRIF 140 assuming only 30% effective usage, the PRIF 140 is a little bit lower but still near to ethanol and better than batteries. Still superior to H2 gas at 700 bar. Again, that's figuring a 70% loss.

PRIF 140 has no emission, at either 30% or 100%. Thus even in a worst case 30% conversion scenario, PRIF 140 is still better than competition, and even more for having no emissions. The phrasing LH2 tank+ refers to a cryo tank. Typically cryo has vessel pressure and safety issues, along with the large energy loss in order to do the freezing. So cryogenic just means that the gas hydrogen has been frozen to negative 273° F. (0°K). That's when it turns liquid. The energy loss in doing that freezing step is incredible, and makes these types of fuel-packages less desirable.

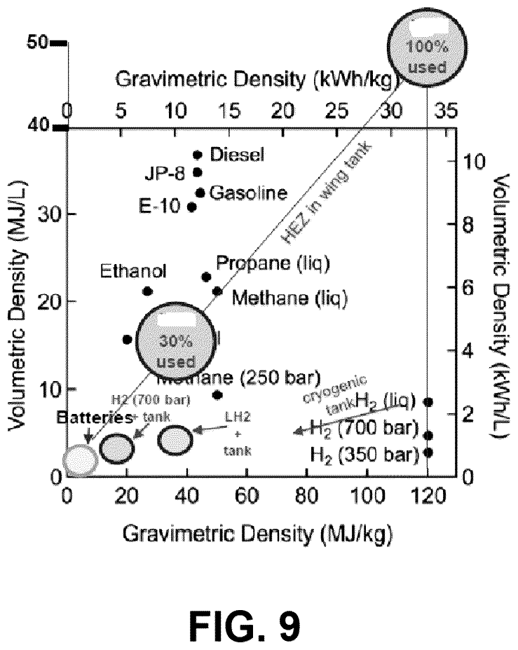

FIG. 9 shows the same energy density comparisons as FIGS. 8A-8B, asserting all the same data but using separate measurement units along the axes. FIG. 9 explains energy potential at 700 bar and 350 bar for other fuels, while PRIF 140 has no pressure. FIG. 9 explains that PRIF 140, even at low efficiency e.g. 30%, is still more effective than the other fuel modalities.
PRIF 140: specific gravity 1.08;
PRIF 140: 35% denser than Jet-A;
PRIF 140: 97-99% hydrogen;
PRIF 140 energy density (at 100% utilization): 47.25 MJ/L, 120 MJ/kg;
PRIF 140 Energy Density (at 30% Utilization): 14.2 MJ/L, 36 MJ/kg;

The comparisons within FIG. 9 are targeted for air travel in which weight and volume (thus density) are important factors. Unlike H2 gas, PRIF 140 can be stored in any type of container. No pressure or cooling is required, and the boiling point of PRIF 140 is 102° F.

Viewing the PRIF 140 in its 100% utilized context, the volumetric and gravimetric densities are much higher than anything else in FIG. 9, including diesel, gas, propane, or methane. Even at 30% utilization, an extremely conservative comparison, the PRIF 140 is still very competitive.

In aviation fuel, one must take into consideration the amount of lift required to lift a fuel to get it up in the air. FIG. 9 focuses on air travel. Diesel weighs 6.8-7 lbs/gallon. PRIF 140 weighs 8.8 pounds/gallons, thus a bit lower on the graph. FIG. 9 show that PRIF 140 is the best pound for pound fuel. Weight matters, including but not limited to a wing tank (normal unpressurized fuel tank located in a wing of a jet aircraft).

FIG. 9 shows that LH2+ plus tank is labeled “plus” thus a cryogenic tank. LH2 liquid hydrogen plus (pressure) tank. The cryogenic tank is the gray one. And you see underneath then H two 700 bar and H two at 350 bar. The differentiation is that the light gray one is cryo and the purple one and the green one are just STP.

Batteries are all the way at the bottom. In the measurements of FIG. 9, batteries come in last in everything.

100% efficiency means total power with absolutely no emissions. Everybody's goal is to be far upper-right in this chart. JP-8 (Jet-A) jet fuel has more potential density, volumetric density power, the highest. Everything invented to date is all clustered around that area.

FIG. 9 points out three circles, showing H2 liquid is the gray, the green is H2 at 700 bar and the pink is H2 at 350 bar, which is the same as batteries. Sub-optimal, if not outright bad.

In working with large industrial sites e.g. Toyota or other, H2 compressed gas and cryogenic tanks cannot be co-located in certain areas where there exist large amounts of liquid-based hydrocarbon fuels. Also, the trucking has to be mitigated to certain weights if heaven forbid, somebody would take that truck and ram it as an act of terrorism. Airports have become mitigated on the potential of terrorist attacks by reducing hydrogen co-located with their other resources. The embodiments of PRIF 140 herein are immune from these types of problems.

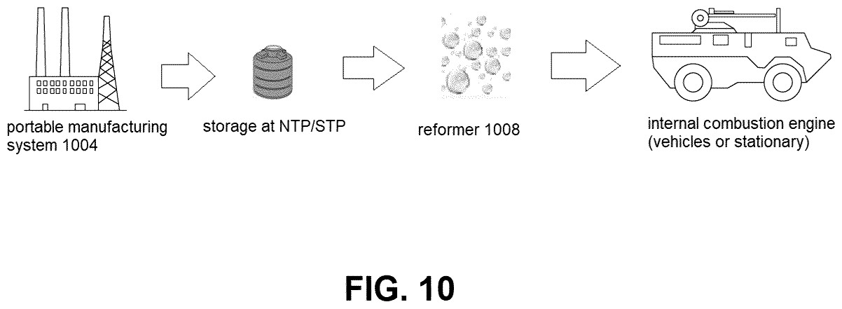

FIG. 10 shows a portable manufacturing system (PMS) 1004 which converts water into PRIF 140 using some form of the proprietary electromagnetic processes shown in FIGS. 1-7. The PMS 1004 can be stood up anywhere that water and at least some electricity are available.

The electricity used to make PRIF 140 is significantly less than the resulting energy content of the stable, safe PRIF 140, and the PRIF 140 can be stored in containers until needed. The energy density of the PRIF 140 created herein is similar to the fuels it replaces. A reformer 1008 can turn the PRIF 140 into H2 in real time for immediate use, or for storage in conventional tanks.

FIG. 10 is important for showing how the embodiments herein facilitate a complete re-thinking of energy distribution and storage, including in hostile environments. One example of this is mobile distribution of PRIF 140 being suitable for military and marine use, partly because PRIF 140 reduces fuel logistics by 90% due to its density.

One can drop PRIF 140 from planes/helicopters and locate it closer to troops with minimal risk. Military folks can't put their diesel storage next to barracks. If someone ignited a piece of C4, they could decimate 10,000 troops. The PRIF 140 has the potential to reduce fuel logistics by as much as 90%.

In a military environment, its most likely any given that internal combustion engine is a diesel, but has the ability to run natural gas. The internal combustion engines (ICE) running natural gas can typically accept hydrogen at near to 100%. The US military runs on diesel. No mini refineries are in the middle east, Syria, etc. Also, the main ingredient of PRIF 140 is water, thus a water-based fuel. If a tote of PRIF 140 lands hard, it do not explode.

After digesting the above, it should be more apparent how the embodiments herein are a major mind-shift and a re-thinking of energy distribution and storage.

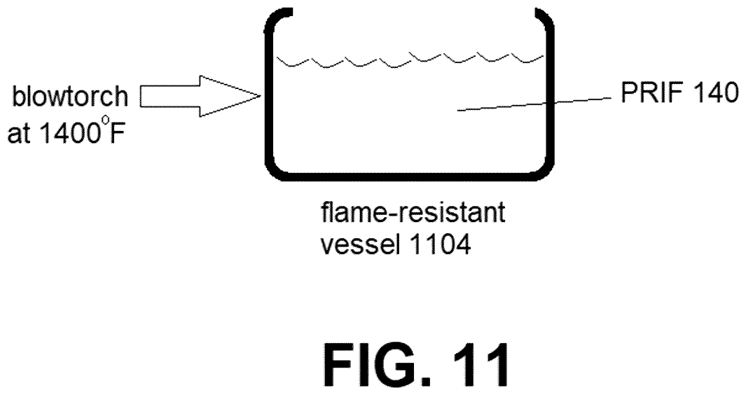

FIG. 11 shows a summary of properties of the PRIF 140. From FIG. 11 it should be apparent that the PRIF 140 is not flammable and its vapor is not flammable. The square glass vessel 1104 simulates a puddle fire suitable for lab testing. Applying this flame for an extended period to prove that PRIF 140 is not flammable in any way, shape or form. Suppose PRIF 140 is in a wing tank with on-board reformer. Now suppose the plane catches fire or has a breach. While that is quite bad, at least the unreformed PRIF 140 won't catch fire.

### PHYSICAL PROPERTIES OF PRIF 140

The PRIF 140 is a clear liquid with a specific gravity of ~1.08, with a pH near zero yet not strongly corrosive. The PRIF 140 is not a solvent for hydrocarbons, and is soluble in water. The PRIF 140 has high electrical conductivity, a freezing point <0° C. and boiling point >100° C.

Additional properties of the PRIF 140 include the following. No greenhouse gases are related in manufacture or use of PRIF 140. Hydrogen is reformed either chemically or electrochemically from PRIF 140. The hydrogen content per cubic meter of PRIF 140 is larger than liquid hydrogen or water. The energy content per cubie meter of PRIF 140 is great than Jet-A. The hydrogen content per kg of PRIF 140 is similar to liquid hydrogen, but with no cryo-storage requirements. PRIF 140 costs less per gallon than Jet-A.

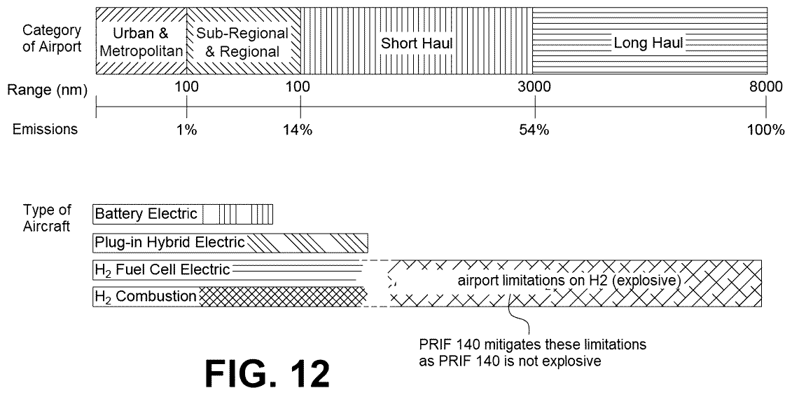

FIG. 12 shows a chart regarding fuel-safety at airports. Fire safety is the single biggest risk of implementing any type of hydrogen-solution, and is a key consideration at airports. Meanwhile, the PRIF 140 is non-flammable and non-toxic, and can be held in wet wing (wing tank) with similar structural detail as current wings. Using an in-wing reformer, H2 can be created “on the fly” for immediate use. Only a small “ready” tank of H2 gas is need.

The blue-transition horizontal bar graph represents size of airport, changing (darkening) as the viewer advances from left to right. The higher green bar chart is already approved on every airport on the planet. The lower blue and purple bar charts represent H2. Airport safety limitations on H2-presence all stop halfway through & turns to checkered background (meaning presently forbidden). The PRIF 140 could remove or supercede this prohibition.

One way to do this is using a wing tank, which is a normal aviation fuel vessel located within a wing. Wing tanks require no pressure, require nothing special, just an enclosure. FIG. 12 safety suggest advantages of taking PRIF 140 that is non-hazardous and then reforming it in mid-air. One gallon of PRIF 140 can extend a plane's travel range, because that one gallon produces so much usable fuel. This image graphed the 30% range for credibility (see FIG. 12), because even sympathetic observers won't believe a hundred percent utilization.

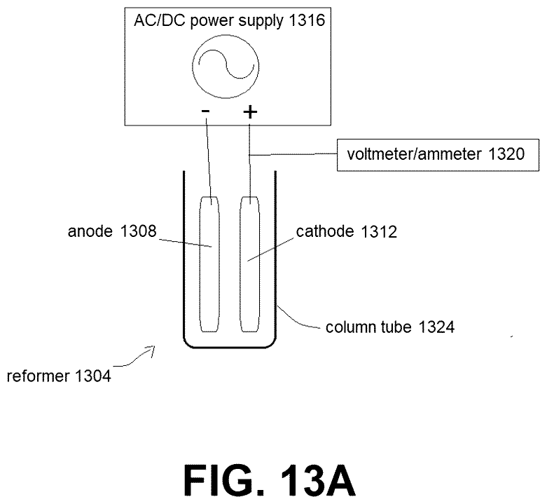

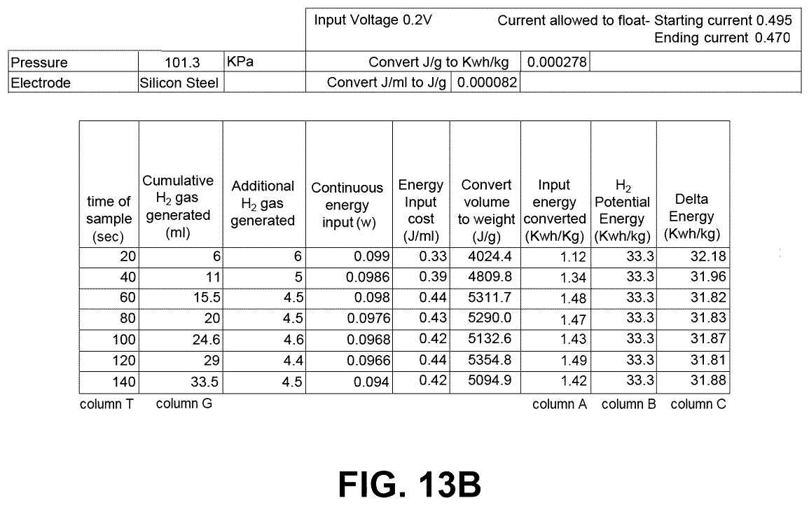

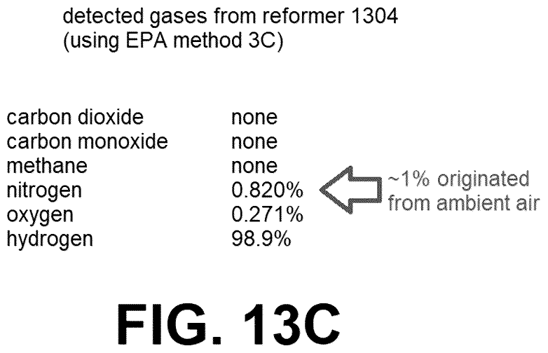

FIG. 13A shows a simplified reformer 1304. However, this visual representation is for example only, and the various embodiments of reformer 1304 discussed herein should not be considered as limited exclusively thereto. The reformer 1304 comprises a column tube 1324 containing two electrodes, an anode 1308 and cathode 1312 connected to a DC power supply 1316. During use, the reformer 1304 is filled with a predetermined amount of PRIF 140, and known amounts of electricity are applied to the anode/cathode 1308/1312 under timed circumstances. Both electrodes are made from silicon steel.

In conventional systems e.g. Hoffman apparati, typically there would be only oxygen (no hydrogen) created on any anode. Meanwhile, the reformer 1304 gets hydrogen from both electrodes.

During use of the reformer 1304, electricity was supplied to the electrodes 1308/1312, which generates gas. Ran the captured gas through a mass gas analyzer (not shown) which captured all gas produced at both the cathode and anode electrodes. An embodiment applied 0.2 volts and 0.49 amps for 20 seconds. This resulted in a hydrogen composition of 98.9%, thereby showing that even with DC power, there is no oxygen being reformed, only hydrogen.

Although not shown in FIG. 13A, there exists a special type of capture-bag to put the gas into, and then run it through a GC mass spec.

FIG. 13B conveys that the embodiments herein can affirm and verify that hydrogen gas is captured at the both anodes and cathodes of the reformer 1304.

A POSITA may not believe that the anode 1308 was making hydrogen (not oxygen), as this is counter-instinctive. Any conventional Hoffman apparatus would have the anode making oxygen-only, going back to when Augustus Hoffman began doing electrolysis of water in 1866. However, for anyone reading this disclosure that may believe there was still oxygen being produced at the anode 1308, FIGS. 13B-13C show how this is not possible.

This is affirmable for several reasons. First, electrolysis is inefficient, energy-eating, and could never be achieved applying on 0.2V. Second, ordinary water is not under consideration here, as the PRIF 140 has components that water does not have. Third, FIG. 13C shows the total gases produced by the reformer 1304. Such production would never be the result of an ordinary electrolysis process.

FIG. 13C is an analysis of all gases produced by the reformer 1304. Its preferable to gas-analyze the reformed hydrogen, due to the trustworthiness and reliability of mass gas analyzers. FIGS. 13B-C effectively prove that both anode and cathode are creating hydrogen. This is possible because the reformer 1304 is not doing electrolysis. FIG. 13C show that there's a 1% air potential for a mis-measurement due to air in the chamber.

FIGS. 13A-B-C also affirm non-electrolysis another way. During the reformation process, the voltmeter 1308 stayed right at 0.2 volts the entire time, which is below any threshold for a successful water-Based electrolysis reaction. This proves there exists no diminishment in delta energy creation, and shows excellent net energy gain throughout the whole system.

FIG. 13B shows other important data. From the time of 20 seconds all the way to 140 seconds (column T) and from 6 ml all the way to 33 ml (column G), FIG. 13B shows there always existed a ~22× energy gain. This is obtained by dividing the number in column B by its corresponding number in column A.

FIG. 13B also shows that the PRIF 140 reaction is not diminishing over time. This is noticeable by viewing column T (time) and column G (gas generated). That in turn affirms that the PRIF 140 is not a mere carrier, it is liquid hydrogen (albeit in a specialized format).

Hydrogen gas yields is 33.3 kilowatt hours per kilogram. Electrical energy cost was ~1.5 kilowatt hours. The reformed gas was >98% hydrogen. Accordingly, it is possible to reform hydrogen where a resulting hydrogen H2 gas volume can contain as much as 22× potential energy as the original amount of energy applied to the PRIF 140.

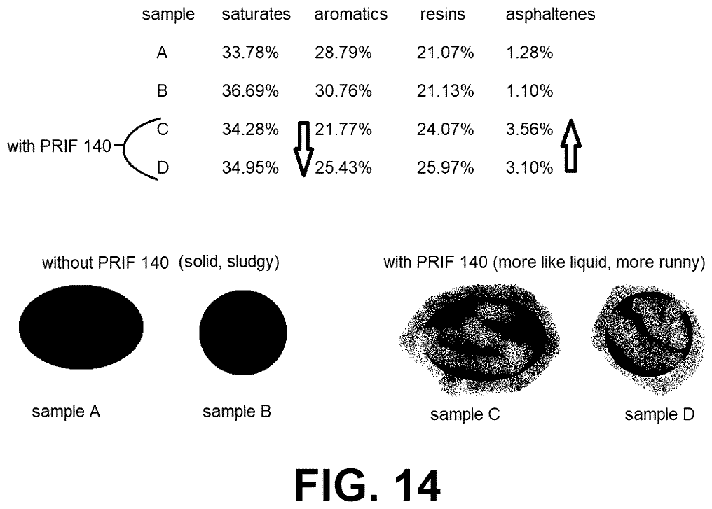

FIG. 14 shows an experiment in which PRIF 140 as added to a sample crude oil, and then performing SARA (Saturated, Aromatics, Resins, Asphaltenes) testing on the resulting combination. SARA is a well-known method of analyzing crude oil, separating its components and classifying those components into four categories.

The specific type of crude used for testing was semi-heavy. In an embodiment, 3 mL of PRIF 140 was added to 30 mL of semi heavy crude and let sit for 72 hours.

Asphaltenes are thick, long chain hydrocarbons. Aromatics are lighter, smaller chains. The PRIF 140 can increase aromatics by degrading the asphaltenes. Earlier, the only way to do that was typically in a refinery having a petroleum cracker, which is inefficient. There exist other polymeric ways of doing this with complex chemistry, but these are expensive and consume too much power.

Meanwhile, the testing described herein proves and affirms that addition of PRIF 140 to crude can break asphaltenes and turn them into aromatics. That is, within any crude composition, a way to decrease asphaltenes is usually good. Addition of PRIF 140 breaks the asphaltenes (bad) into smaller aromatics (good).

FIG. 14 can also be viewed in a context of surface tension and viscosity. The A and B samples appear as a glop that is not spreading. Meanwhile, the C and D samples are spreading due to being thinner, more runny, having lower viscosity. The thicker a sample, the more it stays in unison, due to the aromatics (bad). The thinner it is, the more spreadability it has, due to the asphaltenes (good).

As such, the spreading is significant because the thinner it is, the more spreadable it is, the more valuable due to higher API which means the hydrocarbons contained therein are more accessible for energy production.

IOW, the thinner the oil, the more valuable, because more refinement can be obtained. A thicker oil may result in a weaker refinement rate. The thinner has a stronger refinement rate, which means the thinner the oil, the higher the API rating.

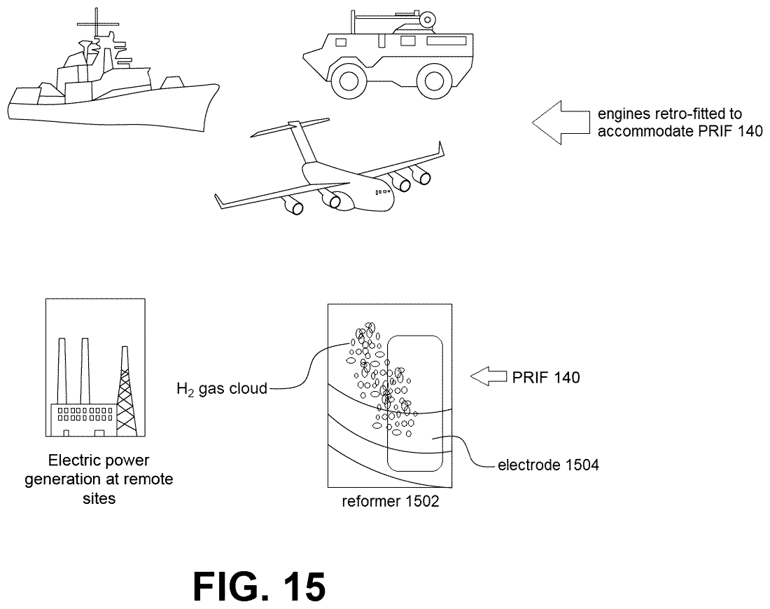

FIG. 15 shows a variety of portable reformer-environments, and includes an actual reformer 1502. The word “reformer” is more accurate and better than “vaporizer” because this semantic avoids even suggesting any use of electricity. Meanwhile, “vaporizer” suggests use of electricity, and thus has connotations not always met by Applicant's various reformers, some of which do not need electricity at all. Applicant's reform process can be performed with minimal and sometimes zero electricity, mostly applied to the measurement meters and MGAs. This includes doing reforming directly inside an aircraft wing as shown in FIG. 15.

PRIF 140 can be formed from liquid into H2 gas chemically, electrically, or a combination of the two. Current reformer designs are capable of 0.7 mW of H2 gas per hour. As shown in FIG. 15, PRIF 140 can be reformed in real-time, via a conventional power plant. It is possible to reform PRIF 140 into hydrogen using less energy than resulting hydrogen energy gain value (again, repeating from earlier, achieving ~22× energy gain).

Using chemical reforming (iron rection), it is possible to make H2 using only ambient temperature energy with no electricity. The reaction rate is temperature-dependent.

As will be discussed in more detail elsewhere within this disclosure, it's also possible to reform PRIF 140 within an existing combustion chamber, thereby eliminating need for a separate reformer.

### ELECTRODES WITHIN REFORMERS

Many different reformers are represented herein. These reformers can have a variety of electrodes made from a variety of materials. Silicone steel has 3% silicone, while silicone cast iron has 14% silicone. Silicone acts as a semiconductor inside the matrix of the steel, which reduces its breakdown rate, makes it more durable. Silicone steel reforms well, and also lasts well. Silicone steel is the number one steel used in all transformers across the United States.

However, the embodiments of reformer electrodes herein are not limited to silicon steel/iron. Either copper or aluminum in certain conditions. Using 3-14% silica helps conserve the deterioration of the underlying metal, yet does not impede electrode-effectiveness.

Silica based composites seem to be good, magnesium based composites also. There also exists an MOF (Metal Organic Framework) composite. Can use custom MOFs for electrodes, composite electrodes, synthetic electrodes and metal organic framework electrodes.

This is advantageous for several reasons, only one of which is that surfactants are less expensive than MOFs. Surfactants also can help with corrosive attributes when mixed in certain applications. Applicant has developed a surfactant package that stops corrosion but didn't affect efficacy. Using alloy based chemistry, proper surfactants mitigate negative factors and can use cheaper electrodes.

The reformer at the lower RH corner of FIG. 15 shows a basic electrode 1504 generating gas bubbles 1508. The PRIF 140 can be reformed in real time, and does not require pre-prep (~=parboiling) prior to reform.

In sharp contrast, making hydrogen gas in advance (pre-prep) is expensive, subtracts from Green H certification, and thus starts any reform process at an energy deficit. That is, starting at a net loss (starting in a hole). This results in losing about 40% of the energy gain of reforming. In conventional reforming, if one achieves a megawatt coming out of that reformer, one likely needs almost three megawatts going in. That's bad. So typically parties don't reform H2 gas in real-time because of lacking sufficient power to drive the reformer, along with the lossy power equation.

A power-generating utility must maintain a base amount of energy being created. No utility or other entity can make hydrogen H2 gas and simultaneously maintain a base energy load because the amount of energy consumed to make that H2 gas is too large. Therefore, real time reforming is normally difficult. However, the PRIF 140 can be reformed in real time. Further, the PRIF 140 can be reformed into hydrogen on-site, or even on-aircraft (e.g. wing tank) potentially.

Another advantage is that a reformer is safer, can't be blown up mid-air. The only explosion would be the hydrogen therein, not what's behind it. Conventional reforming arrangements are more dangerous. That's why military generators must typically be some distance away from barracks and human soldiers.

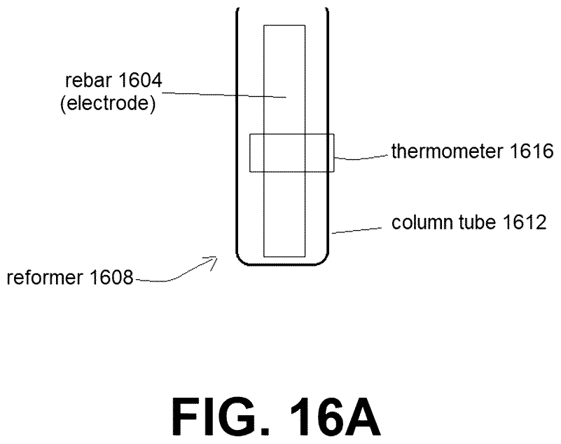

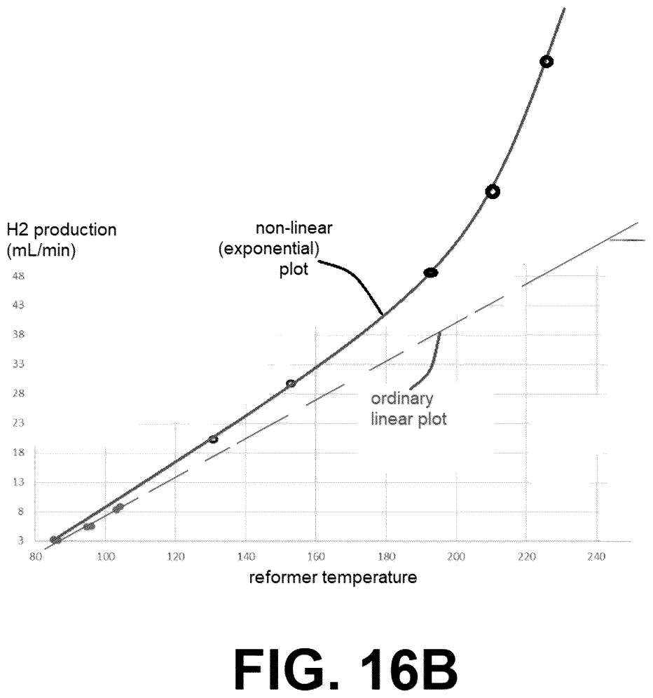

FIG. 16A show a simple reformer 1608 comprising a column tube 1612, a thermometer 1616, and a section 1604 of ordinary rebar acting as an electrode. The PRIF 140 is poured in, not overtly shown in FIG. 16A but assumed to be present. In FIG. 16A, no voltage is applied, and no power source exists. Instead, the re-bar 1604 was pre-heated. Note the voltage is zero, the activation energy originates only from the heat within the rebar 1604, along with characteristics of the PRIF 140. Preheating the rebar 1604, positioning inside the column tube 1612, and then pouring in PRIF 140 and capturing the gas generated thereby (potentially using a capture-bag).

Another important topic is reaction rate, late v. early. Typically when catalyzing, one conventionally starts with the lowest catalyst performer and get to the biggest catalyst performer at the end. This is because as the conventional product catalyzes it becomes less and less reactive.

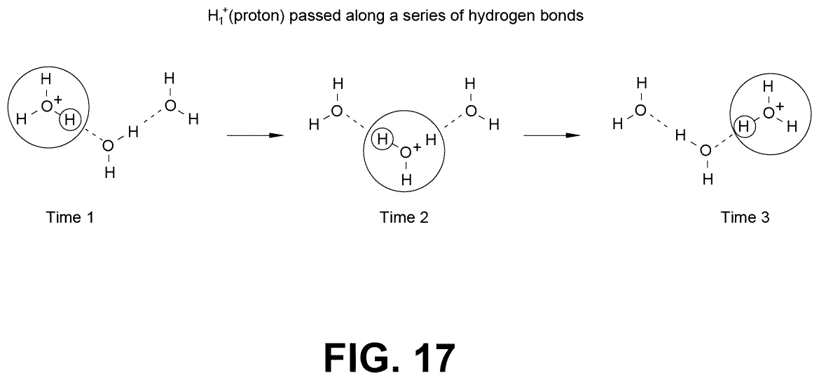

Meanwhile, the embodiments herein achieve the opposite. As shown within FIG. 16B, the PRIF 140 catalytic reaction becomes more violent over time, not less violent. FIG. 16B affirms that the reformation of PRIF 140 is logarithmic both with electrical (e.g. FIG. 13A) and thermal (e.g. FIG. 16A), partly based on the quasi-Grothuss of FIG. 17. The PRIF 140 can achieve almost a 22× power positive if integrating both of them together.

FIG. 16B shows that the reforming is not linear and instead exponential. One reason for this is the Grothuss mechanism which is shown in more detail within FIG. 17.

FIG. 17 conveys that the PRIF 140 also has its own quasi-Grothuss type of behavior. That is, under the right circumstances (such as but not limited to the reformer 1608 of FIG. 16), with the proper levels of activation energy (e.g. 0.2 volts, or zero-voltage using heated rebar), Applicant's H₁+ protons begin tunneling and re-positioning themselves outside of their base location. The structure of the PRIF 140 facilitates this movement, such that the H₁+ protons become easier and easier to release. This in turn means that the reforming discussed herein occurs in an exponential (logarithmic) rate, and not just linear.

There is another advantage of FIGS. 16-17 that is complex to explain. One must take into consideration how many BTUs are being borrowed by the iron rebar 1604 to maintain its specific temperature. Once the reformer 1608 achieves temperature necessary for activation energy, one can use that heated rebar 1604 with its “borrowed” BTUs for vaporizing PRIF 140 inside of a burner at e.g. a steel plant.

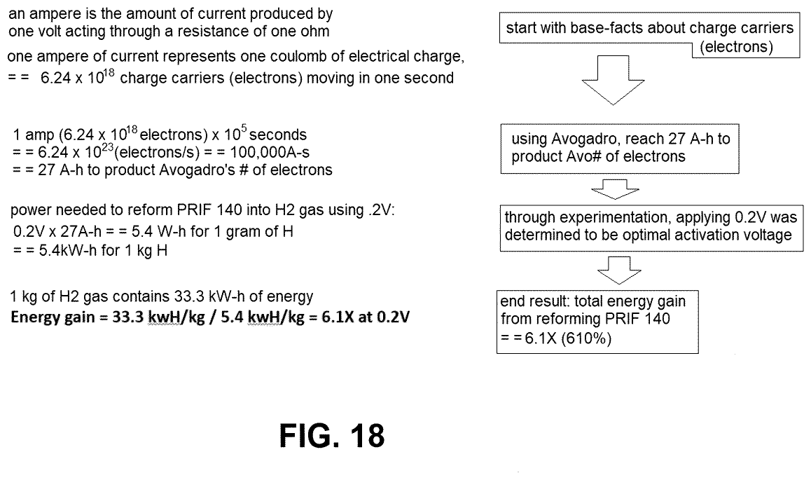

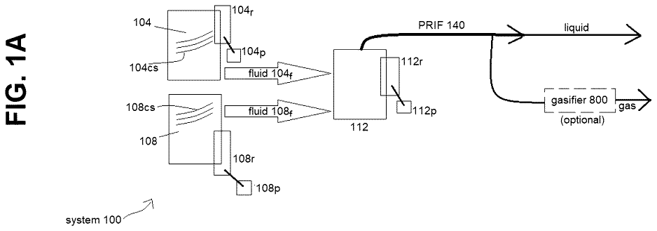

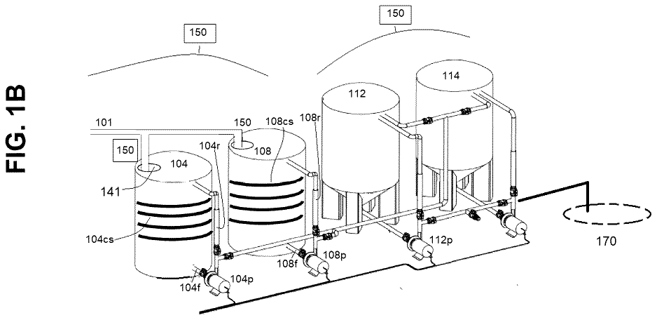

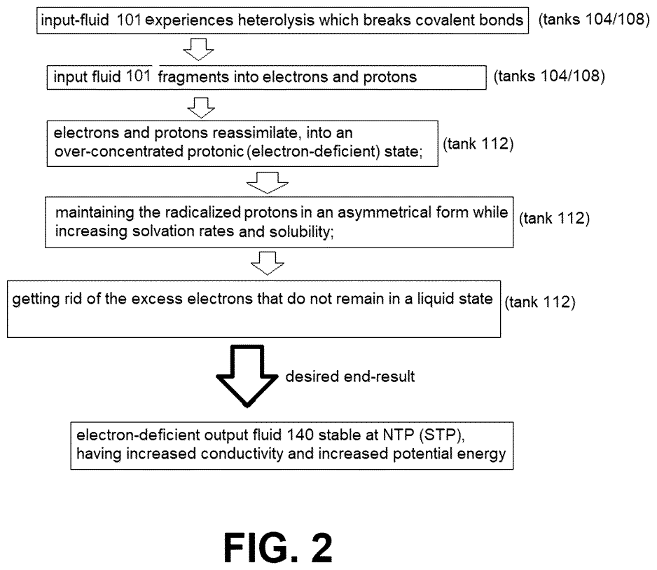

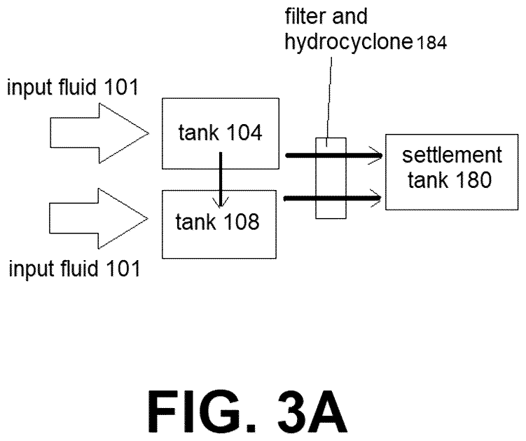

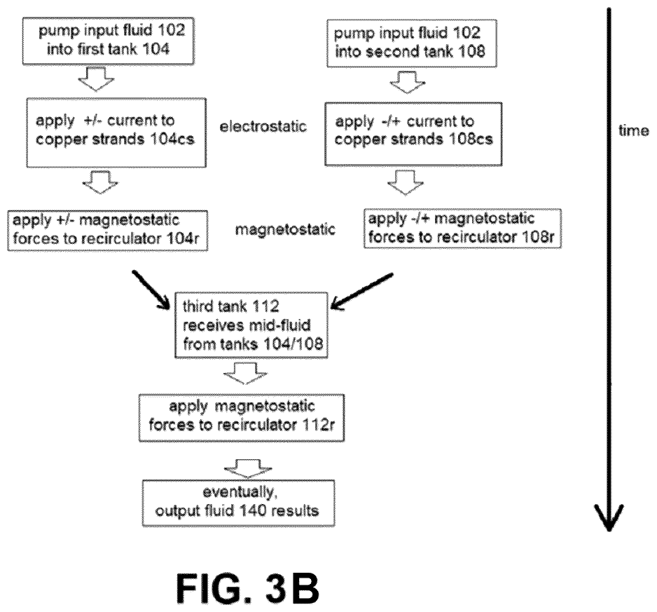

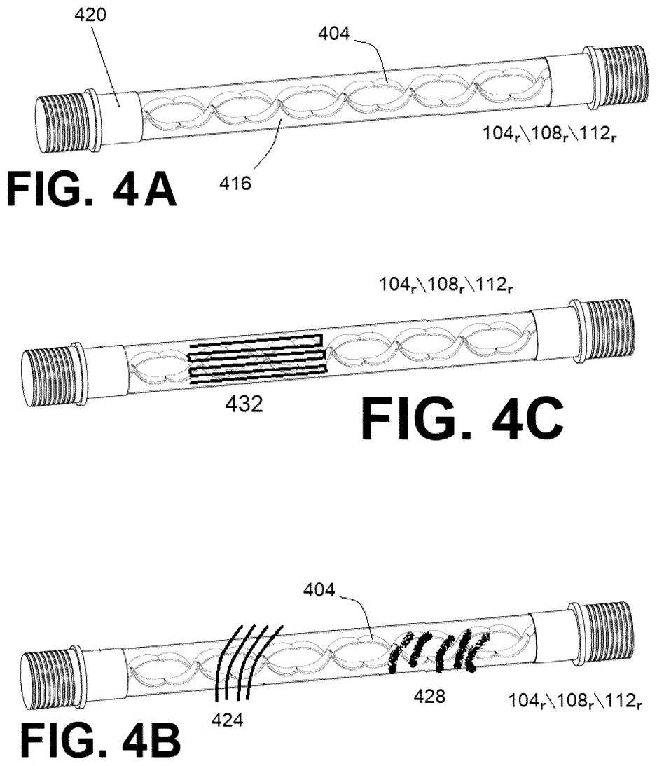

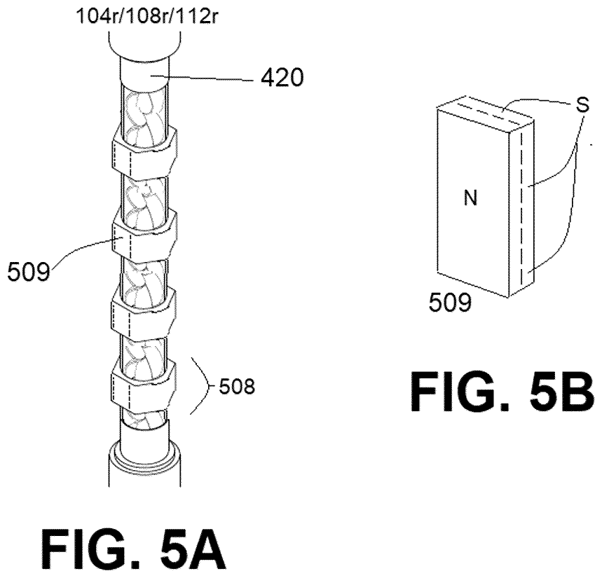

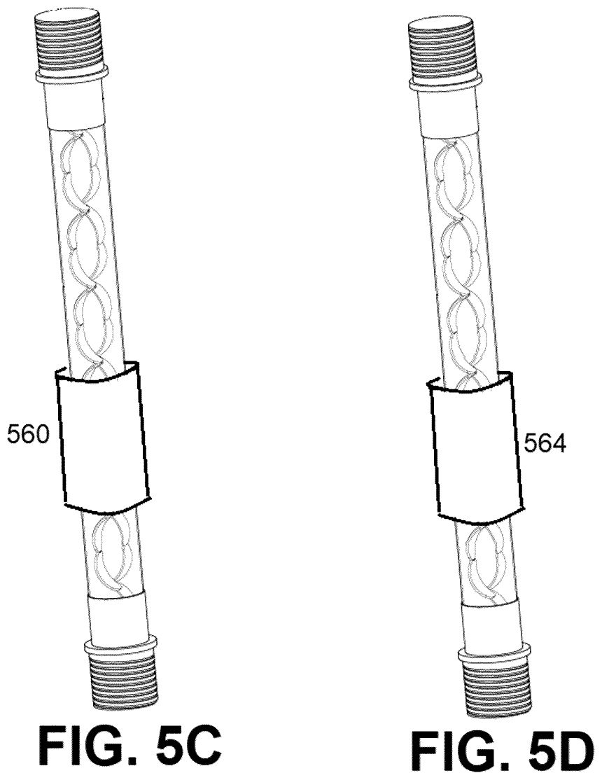

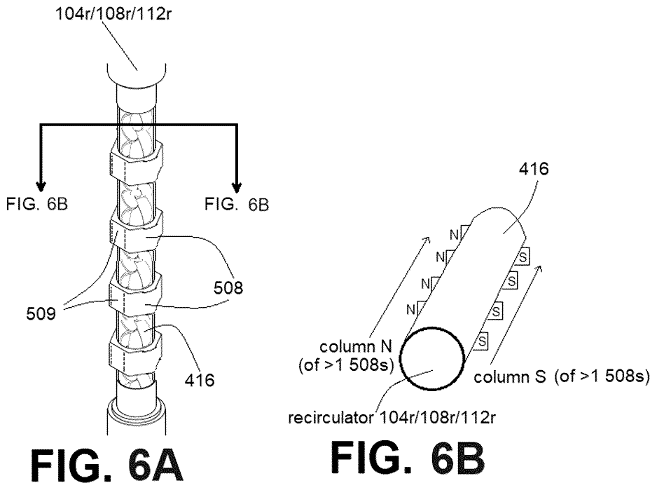

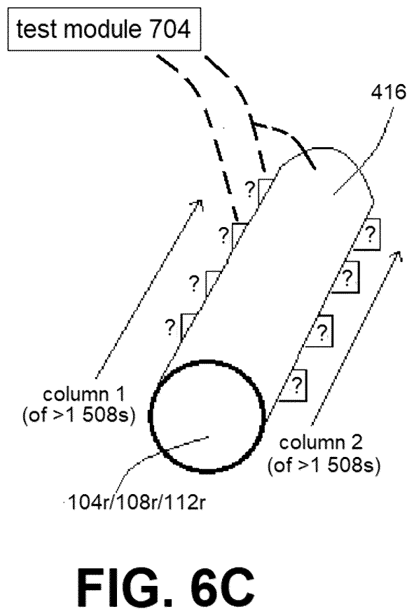

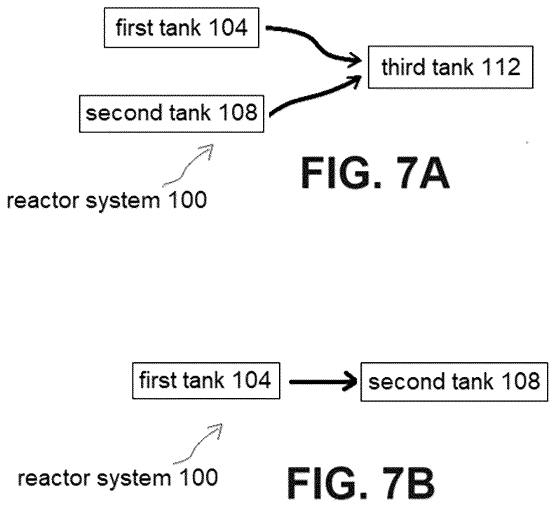

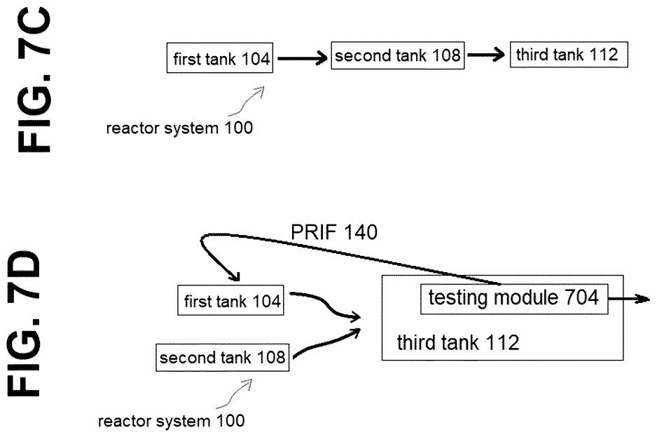

FIG. 18 shows the steps in computing amount of energy needed for creation of 1 kg of H2 gas from the PRIF 140. FIG. 18 uses a voltage supply as an energy source, as opposed to heat, or heated re-bar, or other energy source.

In FIG. 18 one important detail is the red line that has the red equal sign and red arrow. The lowest amount of volts to achieve activation energy was 0.2 volts and then reforming for 27 amp hours because that's how much would be required to make 1 Kg of hydrogen gas.

The PRIF 140 can react effectively with as low as 0.2 volts, and maybe lower. This is sometimes referred to as “activation voltage” and/or “activation energy”. FIG. 18 shows how many electrons are in each one of those potentials showed what the potential lowest energy capability is. A worst case scenario, with no outside help, one would need five kilowatt hours per kilogram to make one kilogram. A worst case scenario you would've a 6× value.

After experimentation it was determined that the best activation voltage is 0.2 volts. FIG. 18 uses this and shows that the PRIF 140 is an extremely efficient hydrogen source.

The PRIF 140 could actually reform at 0.01 volts, although this would be kind of slow, probably not-optimal when high volume production of H2 gas is needed. The point of FIG. 18 is more to show reforming capability. Many remote or adverse locations have limited power available. A low-power low-voltage way of creating energy-rich H2 gas would still have great value, even if (at times) the H2 gas reaction runs a bit slow.

### WRAP-UP

The embodiments herein allow the manufacturer to use high energy density fuel (PRIF 140) anywhere that water is available. Many turbine companies use injection head where the diesel fuel is atomized. They pass diesel fuel through this injector head. The PRIF 140 works just fine in such an injection head, thus could thus be incorporated into a mixed stream blend.

The PRIF 140 is 97-99% hydrogen by volume, and is stable at NTP (STP). The energy density of PRIF 140 is higher than Jet-A (JP-8) on both mass and volume levels. Manufacture of PRIF 140 creates no CO2 emissions.

Finally, H2 gas can be reformed “on the fly” from PRIF 140. This feature eliminates any need for liquid hydrogen or H2 gas to be stored at high pressure or cryo-temperatures. This is very important for safety. Further, existing diesel and turbine engines can be modified to run just fine with an H2 mix.

### DISCLAIMER

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations, or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### APPENDIX A: EXAMPLE HYDROGEN-DONATING INPUT FLUIDS 101

A non-limiting list of potential types of hydrogen-donating fluids can include but is not limited to e.g., HCl—hydrochloric acid, HNO3—nitric acid, H2SO4—sulfuric acid, HBr-hydrobromic acid, HI—hydroiodic acid, HClO4—perchloric acid, HClO3—chloric acid, HO2C2O22H—oxalic acid, H2SO3—sulfurous acid, H20—water, HSO4—hydrogen sulfate ion, H3PO4—phosphoric acid, HNO2—nitrous acid, HF—hydrofluoric acid, HCO2H—methanoic acid, C6H5COOH—benzoic acid, CH3COOH—acetic acid, HCOOH—formic acid, C6H8O7—citric acid, C18H36O2—stearic acid, CH3OH—methyl alcohol, CH3CH2OH—ethyl alcohol, CH3(CH2)3OH—n-butyl alcohol, C3H8O—propanol, CH3CH2CH2OH—n-propyl alcohol, (CH3)3COH—t-butyl alcohol, CH3 (CH2)4OH—n-pentyl alcohol, and (CH3)2CHOH—isopropyl alcohol.

### B-TEAM SPEC-ONLY CLAIM STUBS (part of Spec, not actual Claims)

X. The method of claim X, further comprising: using Avogadro and various computations;
affirming an energy gain of e.g. 22×;
affirming the PRIF 140 to be not a hydrogen carrier, but instead being room temperature liquid hydrogen.

X. The method of claim X, further comprising:
performing a series of tests on various hydrocarbon-based fuels including PRIF 140 in order to determine separate energy densities for each separate component within the group;
plotting them all in the same chart;
plotting the PRIF at both 30% utilization as well as 100% utilization; and
visually establishing an energy density of PRIF 140 compared to the others;

X. The method of claim X, further comprising:
plotting energy potential at 700 bar and 350 bar for various other fuels;
visually conveying that PRIF 140 is stored at NTP/STP;

X. The method of claim X, further comprising:
displaying all plotted energy potentials in a way that aimed at and salient for air travel in which weight and volume are important factors;

X. A method of utilizing a proton-rich fluid in transportation, comprising:
acknowledging that Caterpillar and all the big turbine companies use an “injection head” where the diesel fuel is atomized;
running the PRIF 140 through that same injection head at predetermined concentrations; thereby
incorporating the PRIF 140 into a mixed stream blend.

X. The test-method of claim X, further comprising:
plotting H2 gas outputted from the test-reformer according to temperature applied;
affirming that the plot is logarithmic;
affirming the PRIF 140 catalytic reaction becomes more violent over time, not less violent;

### METHOD OF MANUFACTURING A REACTOR

X. The method of claim X, further comprising:
fabricating one or more electrodes from tungsten;

X. The method of claim X, further comprising:
fabricating the one or more electrodes from one or more composites based on transition metals.

What is claimed is:

1. A method of testing and affirming proton isolation within a proton rich ionic fluid using a test-reformer, comprising:
configuring the test-reformer with one or more containers having one or more electrodes located therein;
locating a predetermined volume of Proton-Rich Ionic Fluid (PRIF) in one or more of the containers;
applying either AC or DC current to the electrodes at predetermined amounts for predetermined times;
capturing all gas generated at the one or more electrodes using a capture-bag;
running all of the captured gas through a mass gas analyzer and measuring for hydrogen and separately for oxygen; and
affirming that the combined gas stream contains less than 5% oxygen by mass.

2. The test-method of claim 1, further comprising:
measuring the combined gas streams and affirming that the reformed hydrogen is between 95%-99% by mass, and the reformed oxygen is less than 3%-5% by mass; and
affirming that hydrogen gas is captured at all of the one or more electrodes of the test-reformer.

3. The test-method of claim 2, further comprising:
measuring all voltage applied to all electrodes; and
affirming that all voltage applied remains below known thresholds for a conventional water-based electrolysis reaction; thereby
affirming that the test-reformer is not doing electrolysis.

4. The test-method of claim 4, further comprising:
affirming a power-out: power-in ratio of 22.

5. The method of claim 4, further comprising:
reforming PRIF into H2 gas chemically.

6. The method of claim 4, further comprising:
reforming PRIF into H2 gas electrically.

7. The method of claim 4, further comprising:
reforming the PRIF into H2 gas both chemically and electrically.

8. The method of claim 4, further comprising:
reforming the PRIF into H2 gas thermally by heating a section of iron first, prior to inserting into the test-reformer; thereby
affirming production of H2 gas via chemical reforming at ambient temperatures where no electricity is applied.

9. The method of claim 4, further comprising:
testing the PRIF with blow-torches and igniters;
affirming that PRIF is non-flammable; thereby
affirming safety and suitability of PRIF for aviation usage and military usage.

10. A method of fabricating a production reformer suitable for reforming a proton rich ionic fluid into H2 gas, comprising:
setting up one or more containers;
the containers having one or more electrodes located therein;
locating a predetermined volume of the proton-rich ionic fluid (PRIF) in one or more of the containers;
applying current to the electrodes at predetermined amounts for predetermined times; and
venting off a produced H2 gas.

11. The method of claim 10, further comprising:
fabricating the one or more electrodes from one or more transition metals.

12. The production-method of claim 10, further comprising:
immediately using any produced H2 gas without storing.

13. The method of claim X, further comprising:
configuring the production reformer to be a mobile, portable device.

14. The production-method of claim 13, further comprising:
the production reforming occurring within a battleship.

15. The production method of claim 13, further comprising:
the production reforming occurring within a jet aircraft wing tank.

16. The production-method of claim 13, further comprising:
the production reforming occurring within a power plant.

17. The method of claim 13, further comprising:
factoring that the US military runs on diesel and does not have diesel refineries near to battlefields;
packaging the PRIF in specialized tuff-tote containers that can be dropped from planes/helicopters; thereby
improving military fuel transportation and military supply-chain logistics.

18. The method of claim 17, further comprising:
configuring the reformer to be activated by inert materials that are easily stored.

19. The method of claim 10, further comprising:
fabricating the one or more electrodes from silicone cast iron having 3-14% silicone.

20. The method of claim 10, further comprising:
fabricating the one or more electrodes from silicone steel having 3-14% silicone.
